(19) United States
(12) Patent Application Publication
(10) Pub. No.: US 2023/0263655 A1
(43) Pub. Date: Aug. 24, 2023

(54) FLUID COLLECTION DEVICES, SYSTEMS AND METHODS

(71) Applicant: PUREWICK CORPORATION, Covington, GA (US)

(72) Inventors: Ashley Marie Johannes, Statham, GA (US); Martyn Mitchell, Hertfordshire (GB); Samuel Edmund Whittome, Cambridge (GB); Leanne Yip Heung Win, Wembley, Middlesex (GB)

(21) Appl. No.: 18/043,807

(22) PCT Filed: Sep. 1, 2021
(86) PCT No.: PCT/US2021/048661

§ 371 Date: Mar. 2, 2023

Related U.S. Application Data
(60) Provisional application No. 63/074,051, filed on Sep. 3, 2020.

(51) Int. Cl. A61F 5/455

(52) U.S. Cl. CPC A61F 5/455

(57) ABSTRACT

A fluid collection device may include a fluid impermeable barrier and a fluid permeable body in some examples. The fluid impermeable barrier includes an inner surface at least partially defining a chamber. The fluid impermeable barrier also defines an opening to the chamber extending longitudinally along the fluid impermeable barrier. The chamber may be configured to accept a fluid permeable body. The fluid permeable body may be at least partially exposed by the opening defined by the fluid impermeable barrier and configured to wick fluid from a surface of the fluid permeable body into the chamber. In some examples, the fluid permeable body may include a fluid permeable membrane and a fluid permeable support. In some examples, the fluid permeable membrane may be omitted. In some examples, the fluid permeable support may include at least a portion that is not fluid permeable.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers, spills, and other hygiene issues. Urinary catheters be can be uncomfortable, painful, and can cause urinary tract infections.

Thus, users and manufacturers of fluid collection devices continue to seek new and improved devices, systems, and methods to collect urine.

### SUMMARY

Embodiments disclosed herein are fluid collection devices, fluid collection systems, and components thereof.

In some embodiments, a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra, and a fluid permeable body positioned at least partially within the chamber to extend across at least a portion of the opening and configured to wick fluid away from the opening, wherein the fluid permeable body comprises a hydrophilic fluid permeable membrane and a support at least partially covered by the hydrophilic fluid permeable membrane. Technical advantages of the hydrophilic fluid permeable membrane may include improved movement of fluid through the fluid permeable support, removal of fluid from the fluid collection device, reduce a risk of leaks, and/or reduce feelings of wetness by a wearer.

In some embodiments, a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra, and a fluid permeable body positioned at least partially within the chamber to extend across at least a portion of the opening and configured to wick fluid away from the opening, wherein the fluid permeable body comprises a support at least partially covered by a fluid permeable membrane including a wicking fabric, wherein the wicking fabric is configured to wick the fluid from an outer surface of the fluid permeable membrane to an inner surface of the fluid permeable membrane. Technical advantages of the wicking fabric may include providing more comfort (e.g., less chafing, soft feeling) to a wearer of the fluid collection device.

In some embodiments, a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra, and a fluid permeable body positioned at least partially within the chamber to extend across at least a portion of the opening and configured to wick fluid away from the opening, wherein the fluid permeable body comprises a support comprising a wicking portion proximate the opening and a packing portion proximate an interior surface of the chamber. Technical advantages of the packing portion may include better fluid draining . The packing portion may also allow suction from a vacuum source to be concentrated, which may improve removal of fluid from the fluid collection device

In some embodiments, a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra, and a fluid permeable body positioned at least partially within the chamber to extend across at least a portion of the opening and configured to wick fluid away from the opening. The fluid permeable body may include a support including a wicking portion proximate the opening, a packing portion proximate an interior surface of the chamber, and a sparger tube disposed in a center of the fluid permeable body, wherein the sparger tube extends at least a length of the fluid permeable body along a long axis of the fluid permeable body. Technical advantages of the sparger tube include equilibrium of pressure along a length of the sparger tube. This may allow fluids to be collected along a greater length of the tube rather than having to be transmitted to a reservoir prior to being drawn into the conduit.

In some embodiments, a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra, and a fluid permeable body positioned at least partially within the chamber to extend across at least a portion of the opening and configured to wick fluid away from the opening, wherein the fluid permeable body comprises a support comprising a first layer and a second layer disposed concentrically about the first layer, wherein the first layer and the second layer have at least one of a different porosity or density. Technical advantages of layers with different porosity or density may include equilibration of pressure along a length of the fluid permeable body. This may permit better fluid wicking along a length of the fluid permeable body and/or make it easier for light fluid flows to break the surface tension of the fluid permeable membrane and/or outer layer. A technical advantage may further include improved wicking and/or reduced surface tension, which may reduce dependence on anatomical fit and/or placement for proper performance of the fluid collection device.

In some embodiments, a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra and a fluid permeable body positioned at least partially within the chamber to extend across at least a portion of the opening and configured to wick fluid away from the opening, wherein the fluid permeable body comprises a support comprising a material having at least one of a density or a porosity gradient along a short axis of the fluid permeable body. Technical advantages of a density or a porosity gradient may include equilibratation of pressure along a length of the fluid permeable body. This may permit better fluid wicking along a length of the fluid permeable body and/or make it easier for light fluid flows to break the surface tension of the fluid permeable membrane and/or fluid permeable support. Technical advantages of improved wicking and/or reduced surface tension may include reduced dependence on anatomical fit and/or placement for proper performance of the fluid collection device.

In some embodiments, a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra, and a fluid permeable body positioned at least partially within the chamber to extend across at least a portion of the opening and configured to wick fluid away from the opening, wherein the fluid permeable body comprises a support comprising a material having at least one of a density or a porosity gradient along a long axis of the fluid permeable body. Technical advantages of a density or a porosity gradient along the long axis may include greater capacity for managing bolus flow (e.g., less likely to experience runoff and/or over saturation) and drawing the fluid into the fluid collection deviceand away from the wearer as well as condensing the flow path of fluid and/or air, which may encourage fluid flow to the reservoir where the fluid can be removed from the fluid collection device by the conduit.

In some embodiments, a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra, and a fluid permeable body positioned at least partially within the chamber to extend across at least a portion of the opening and configured to wick fluid away from the opening, wherein the fluid permeable body comprises a fluid permeable membrane comprising a plurality of slot valves. Technical advantages of the slot valves may include accommodation of a bolus flow of fluid.

In some embodiments, a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra, and a fluid permeable body positioned at least partially within the chamber to extend across at least a portion of the opening and configured to wick fluid away from the opening, wherein the fluid permeable body comprises a support comprising a plurality of spokes extending from a central portion of the fluid permeable body to an outer perimeter of the fluid permeable body along a short axis of the fluid permeable body, wherein the plurality of spokes define a plurality of spaces there between. Technical advantages of the support may include equilibration of pressure along a length of the fluid permeable body, concentrating the airflow and/or fluid flow to permit more focused (e.g., stronger) suction, and/or encouragement fluids to remain concentrated (e.g., not spread out through the fluid permeable body), which may improve fluid flow towards the reservoir and/or into the conduit.

In some embodiments, a fluid collection device may include a fluid impermeable barrier having a concave inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a urethra, wherein a perimeter of the opening defines two opposing U-type shaped portions coupled at an angle. Technical advantages of the fluid collection device may include the ability to use the fluid collection device with both male and female wearers.

The technical advantages disclosed are provided merely as examples, and the embodiments disclosed herein may provide additional or alternative technical advantages in some applications. Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric front view of a female fluid collection device according to at least one embodiment of the disclosure.

FIG. 1B is an exploded isometric view of the female fluid collection device of FIG. 1A.

FIGS. 2A and 2B are cross-sectional views of the female fluid collection device of FIG. 1A taken along line 2-2 thereof according to at least one embodiment of the disclosure.

FIG. 3 is a cross-sectional view of the fluid collection device taken along line 3-3 of FIG. 1A according to at least one embodiment of the disclosure.

FIG. 4 is a schematic illustration of system that includes a fluid collection device according to at least one embodiment of the disclosure.

FIG. 5 is a cross-sectional view along a long axis of a fluid collection device including a fluid impermeable barrier and a fluid permeable body according to at least one embodiment of the disclosure.

FIG. 6 is a cross-sectional view along a long axis of a fluid collection device including a fluid impermeable barrier and a fluid permeable body according to at least one embodiment of the disclosure.

FIG. 7A is a cross-sectional view along a long axis of a fluid collection device 700 including a fluid impermeable barrier and a fluid permeable body according to at least one embodiment of the disclosure.

FIG. 7B is a cross-sectional view along a short axis of the fluid collection device of FIG. 7A.

FIG. 8A is a cross-sectional view along a long axis of a fluid collection device including a fluid impermeable barrier and a fluid permeable body according to at least one embodiment of the disclosure.

FIG. 8B is a cross-sectional view along a short axis of the fluid collection device of FIG. 8A.

FIG. 9A is a cross-sectional view along a long axis of a fluid collection device including a fluid impermeable barrier and a fluid permeable body according to at least one embodiment of the disclosure.

FIG. 9B is a cross-sectional view along a short axis of the fluid permeable body of the fluid collection device of FIG. 9A.

FIG. 10A is a cross-sectional view along a long axis of a fluid collection device including a fluid impermeable barrier and a fluid permeable body according to at least one embodiment of the disclosure.

FIG. 10B is a cross-section along a short axis of the fluid permeable body of the fluid collection device of FIG. 10A.

FIG. 11 is a cross-sectional view along a long axis of a fluid collection device including a fluid impermeable barrier and a fluid permeable body according to at least one embodiment of the disclosure.

FIG. 12A is a cross-sectional view along a long axis of a fluid collection device including a fluid impermeable barrier and a fluid permeable body according to at least one embodiment of the disclosure.

FIGS. 12B and 12C illustrate the operation of the slot valve of the fluid permeable body of FIG. 12A.

FIG. 13A is a view of a fluid collection device including a fluid impermeable barrier and a fluid permeable body according to at least one embodiment of the disclosure.

FIG. 13B is a cross-sectional view of the fluid permeable body along a short axis of the fluid collection device of FIG. 13A.

FIG. 14 is a view of a fluid collection device according to at least one embodiment of the present disclosure.

### DETAILED DESCRIPTION

Embodiments disclosed herein are directed to fluid collection devices and portions thereof. The fluid collection devices disclosed herein are configured to collect fluids from an individual. The fluids collected by the fluid collection devices can include urine. The fluids collected by the fluid collection devices can also include at least one of vaginal discharge, penile discharge, reproductive fluids, blood, sweat, or other bodily fluids.

The fluid collection devices may include one or more components for drawing (e.g., wicking) fluid from a wearer and/or a surface of the fluid collection device into an interior of the fluid collection device. In some embodiments, the fluid collection devices may include a fluid permeable body for wicking fluid into the fluid collection device. In some embodiments, the fluid permeable body may include a fluid permeable membrane at least partially surrounding a support. In some embodiments, the support may be a fluid permeable support. In some embodiments, the fluid collection device may include a fluid permeable support without a fluid permeable membrane. The support alone or in combination with the fluid permeable membrane may direct fluids to an interior of the fluid collection device where the fluid may be removed from the fluid collection device.

As described herein, the fluid permeable membrane and/or support may include one or more of a variety of materials such as nonwoven fabrics, foams, polymers, and/or meshes. The fluid permeable membrane and/or support may include one or more of a variety of structures and/or material properties. For example, in some embodiments, the fluid permeable membrane may include tapered pores and/or slot valves. In some embodiments, the fluid permeable membrane may be hydrophilic on at least one surface. In some embodiments, the support may have a porosity and/or density that has a gradient along a long axis or a short axis of the fluid permeable body. In some embodiments, the support may include one or more spokes that direct air and/or fluid flow between spaces between the spokes. In some embodiments, the support may include a reverse Sparger. In some embodiments, the support may include a combination of wicking and non-wicking (e.g., packing) materials.

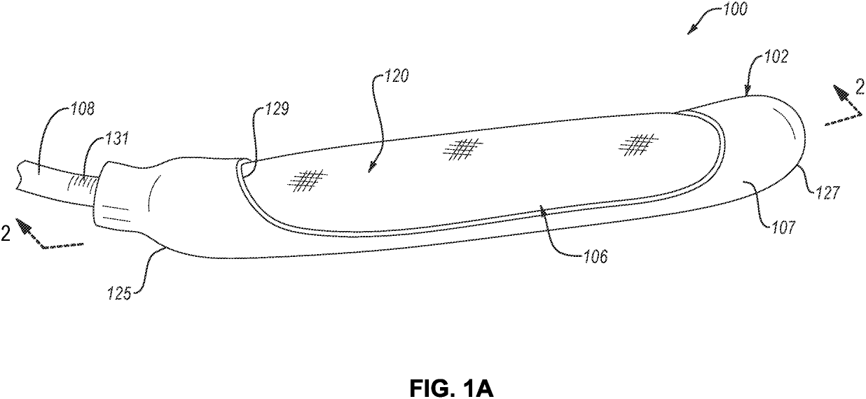

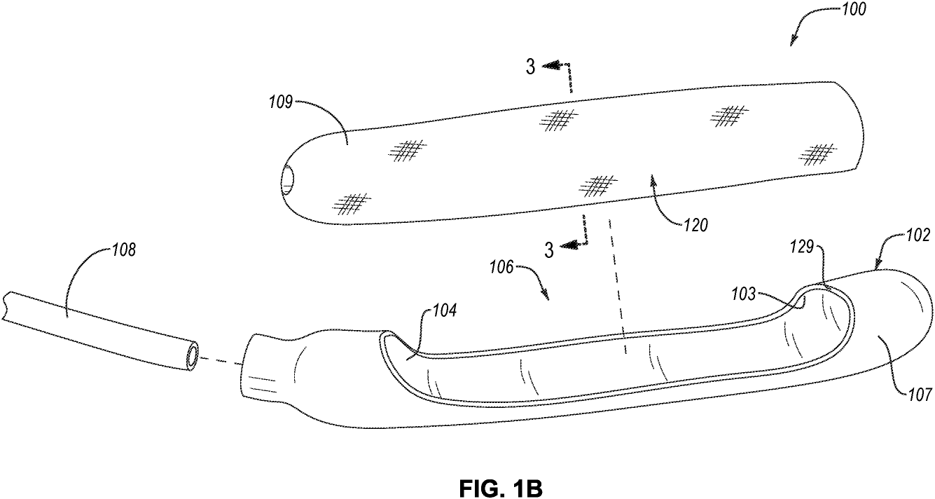

FIG. 1A is an isometric view of a fluid collection device 100 according to at least one embodiment of the disclosure. The fluid collection device 100 is an example of a female fluid collection device 100 that is configured to receive fluids from a female wearer. The fluid collection device 100 includes a fluid impermeable barrier 102 having a first end region 125 and a second end region 127. The first end region 125 may include an aperture 124 for accepting a conduit 108 that may couple the fluid collection device 100 to a vacuum source (e.g., see vacuum device 470). The fluid impermeable barrier 102 at least partially defines a chamber 104 (e.g., interior region, shown in FIG. 1B) and includes an inward border or edge 129 defining an opening 106. The fluid impermeable barrier 102 may be substantially cylindrical in shape between the first end region 125 and the second end region 127. The opening 106 is formed in and extends through the fluid impermeable barrier 102, thereby enabling fluids to enter the chamber 104 from outside of the fluid collection device 100. The opening 106 can be configured to be positioned adjacent to a female urethra in some applications.

In some applications, the fluid collection device 100 may be positioned proximate to the female urethra and urine may enter the interior region of the fluid collection device 100 via the opening 106. The fluid collection device 100 is configured to receive the fluids into the chamber 104 via the opening 106. For example, the opening 106 can exhibit an elongated shape that is configured to extend from a first location below the urethral opening (e.g., at or near the anus or the vaginal opening) to a second location above the urethral opening (e.g., at or near the clitoris or the pubic hair). The opening 106 can exhibit an elongated shape since the space between the legs of a female wearer is relatively small when the legs of the female wearer are closed, thereby only permitting the flow of the fluids along a path that corresponds to the elongated shape of the opening 106. For example, the opening 106 can extend longitudinally along the fluid impermeable barrier. The opening 106 in the fluid impermeable barrier 102 can exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 can exhibit a width that is greater than 50% of the circumference of the fluid collection device 100 since a vacuum (e.g., suction) through a conduit 108 pulls the fluid into the conduit 108. In some embodiments, the opening 106 may be vertically oriented (e.g., having a major axis parallel to the longitudinal axis of the device 100). In some embodiments (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 can include two opposing arced portions, the arcs following the outer circumference or periphery of the substantially cylindrical fluid impermeable barrier 102.

The fluid impermeable barrier 102 may also temporarily store the fluids in the chamber 104. As such, the fluid impermeable barrier 102 substantially prevents the fluids from exiting the portions of the chamber 104 that are spaced from the opening 106. The fluid impermeable barrier 102 may be flexible, allowing the fluid collection device 100 to bend or curve when positioned against the body of a wearer. For example, the fluid impermeable barrier 102 can be formed of any suitable fluid impermeable materials, such as a fluid impermeable polymer (e.g., silicone, polypropylene, polyethylene, polyethylene terephthalate, a polycarbonate, etc.), polyurethane films, TPE, oil, another suitable material, or combinations thereof. In some embodiments, the fluid impermeable barrier 102 can include one or more thermoplastic elastomers. The one or more thermoplastic elastomers may be combined with at least one of silicone and oil. In many embodiments, the fluid impermeable barrier 102 can include a composition having at least silicone and oil therein.

The fluid collection device 100 can include a fluid permeable body 120 or layer disposed in the chamber 104. The fluid permeable body 120 can cover or extend across at least a portion (e.g., all) of the opening 106. The fluid permeable body 120 can be configured to wick any fluid away from the opening 106, thereby preventing the fluid from escaping the chamber 104. The fluid permeable body 120 also can wick the fluid generally towards an interior of the chamber 104, as discussed in more detail below. A portion of the fluid permeable body 120 can define a portion of an outer surface of the fluid collection device 100. Specifically, the portion of the fluid permeable body 120 defining the portion of the outer surface of the fluid collection device 100 can be the portion of the fluid permeable body 120 exposed by the opening 106 defined by the fluid impermeable barrier 102. At least a portion of the fluid permeable body 120 exposed by the opening 106 may contact the wearer.

The fluid permeable body 120 can include any material that can wick the fluid. The permeable properties referred to herein can be wicking, capillary action, diffusion, or other similar properties or processes, and are referred to herein as “permeable” and/or “wicking.” Such “wicking” may exclude absorption into the wicking material (e.g., retention of fluid by the material). Put another way, substantially no absorption of fluid in the material may take place after the material is exposed to the fluid and removed from the fluid for a time. While no absorption is desired, the term “substantially no absorption” may allow for nominal amounts of absorption of fluid into the wicking material (e.g., absorbency), such as less than about 10 wt% of the dry weight of the wicking material, less than about 7 wt%, less than about 5 wt%, less than about 3 wt%, less than about 2 wt%, less than about 1 wt%, or less than about 0.5 wt% of the dry weight of the wicking material.

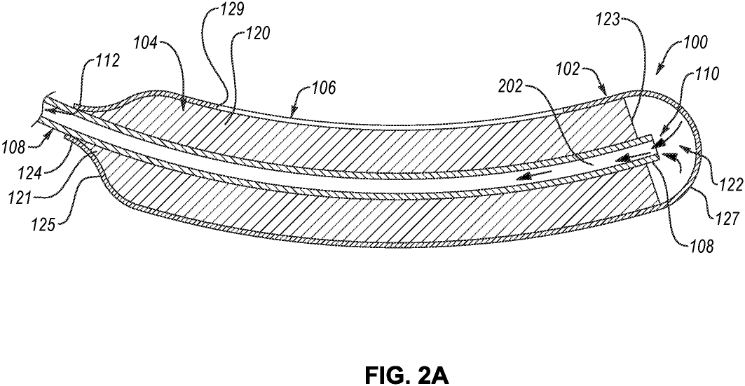

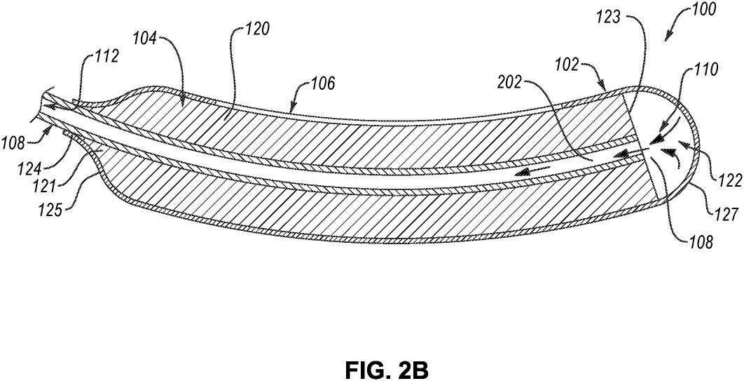

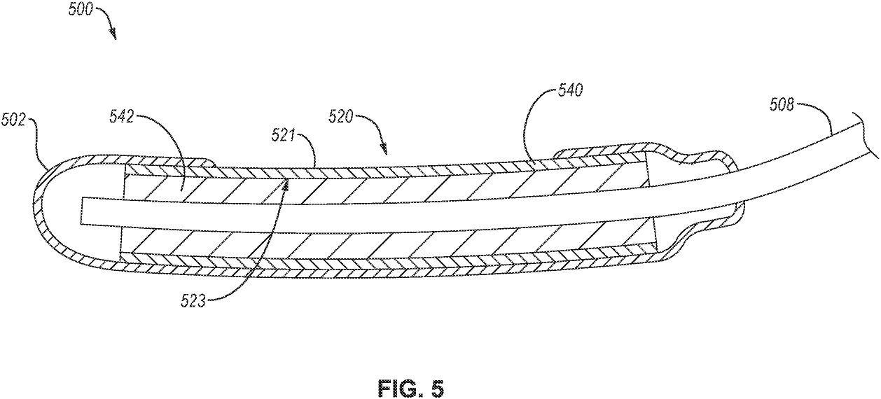

The fluid permeable body 120 can include a one-way fluid movement material. As such, the fluid permeable body 120 can remove fluid from the area around the female urethra, thereby leaving the area dry. The fluid permeable body 120 can enable the fluid to flow generally towards a reservoir 122 (shown in FIGS. 2A and 2B) of void space formed within the chamber 104. For example, the fluid permeable body 120 can include a porous or fibrous material, such as hydrophilic polyolefin. Examples of polyolefin that can be used in the fluid permeable body 120 include, but are not limited to, polyethylene, polypropylene, polyisobutylene, ethylene propylene rubber, ethylene propylene diene monomer, or combinations thereof. The porous or fibrous material can be extruded into a substantially cylindrically shape to fit within the chamber 104 of the fluid impermeable barrier 102. The fluid permeable body 120 can include varying densities or dimensions, for example as described in more detail with reference to FIGS. 5-14. Moreover, the fluid permeable body 120 can be manufactured according to various manufacturing methods, such as molding, extrusion, or sintering.

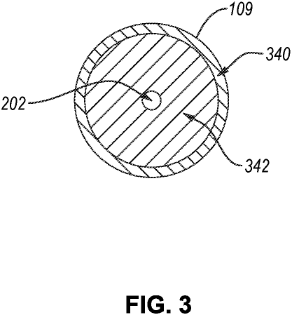

In some embodiments, during use, the fluid permeable body 120 extends from the conduit 108 to interface the fluid impermeable barrier 102 and the opening 106. In some embodiments, a majority of the outer surface 109 (shown in FIG. 1B and FIG. 3) of the fluid permeable body 120 interfaces with an inner surface 103 (shown in FIG. 1B) of the fluid impermeable barrier 102. In other embodiments, a majority of the outer surface 109 may be exposed by the opening 106 of the fluid impermeable barrier 102. In some embodiments, at least a portion of the fluid permeable body 120 extends continuously between the opening 106 and the reservoir 122 to wick any fluid from the opening 106 directly to the reservoir 122. Moreover, when the fluid impermeable barrier is flexible and the fluid permeable body 120 is configured to wick fluid from the body rather than absorb fluid from the body and hold the fluid against the body, the fluid collection device 100, in some embodiments, is free from a seal or cushioning ring on the inward edge 129 defining the opening 106.

FIG. 3 is a cross-sectional view of the fluid collection device 100 taken along line 3-3 of FIG. 1A. As shown in FIG. 3, the fluid permeable body 120 can include a fluid permeable membrane 340 covering or wrapped around at least a portion of a fluid permeable support 342, with both the fluid permeable membrane 340 and the fluid permeable support 342 being disposed in the chamber 104. The fluid permeable membrane 340 can cover or extend across at least a portion (e.g., all) of the opening 106. The fluid permeable membrane 340 can be configured to wick any fluid away from the opening 106, thereby preventing the fluid from escaping the chamber 104. In some embodiments, at least one of the fluid permeable membrane 340 or the fluid permeable support 342 include nylon configured to wick fluid away from the opening 106. The material of the fluid permeable membrane 340 and the fluid permeable support 342 also can include natural fibers. In such examples, the material may have a coating to prevent or limit absorption of fluid into the material, such as a water repellent (e.g., hydrophobic) coating. In some embodiments, the fluid permeable membrane 340 may be hydrophobic to prevent/limit absorption of fluid due to the chemical properties of the material(s) included in the fluid permeable membrane 340 and/or due to the shape and/or weave of the fluid permeable membrane 340.

The fluid permeable membrane 340 can also wick the fluid generally towards an interior of the chamber 104, as discussed in more detail below. The fluid permeable membrane 340 can include any material that can wick the fluid. For example, the fluid permeable membrane 340 can include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane 340 from gauze, soft fabric, and/or smooth fabric can improve comfort of the wearer, for example by reducing chaffing of the wearer’s skin caused by the fluid collection device 100. Other embodiments of fluid permeable bodies 120, fluid permeable membranes 340, and/or fluid permeable supports 342, are described in more detail herein, for example, in reference to FIGS. 5-14. In some embodiments, the fluid permeable body 120 includes a fluid permeable support 342 including a porous nylon structure and a fluid permeable membrane 340 including gauze.

The fluid permeable body 120 is disposed within a chamber 104 (shown in FIGS. 2A and 2B) of the fluid impermeable barrier 102 of the fluid collection device 100 and is exposed to the urethra of the user 150 through the opening 106 in the fluid collection device 100. Fluids received in the chamber 104 of the fluid collection device 100 from the urethra can be removed through the conduit 108.

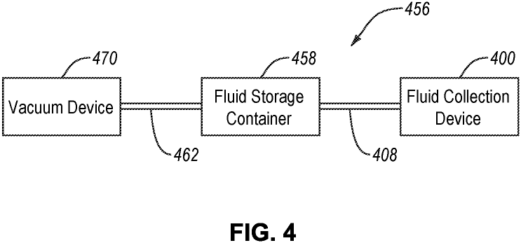

FIG. 2A is a cross-sectional view of the fluid collection device 100 taken along line 2-2 of FIG. 1A. The fluid collection device 100 also includes conduit 108 that is at least partially disposed in the chamber 104. The conduit 108 (e.g., a tube) includes an inlet 110 at a second end region 127 of the fluid impermeable barrier 102 and an outlet 112 at a first end region 125 of the fluid impermeable barrier 102 positioned downstream from the inlet 110. The conduit 108 provides fluid communication between an interior region of the chamber 104 and a fluid storage container (e.g., fluid storage container 458 shown in FIG. 4) or a portable vacuum source (e.g., vacuum device 470 shown in FIG. 4). For example, the conduit 108 may directly or indirectly fluidly couple the interior region of the chamber 104 and/or the reservoir 122 with the fluid storage container or the portable vacuum source.

In the illustrated embodiment, the fluid permeable body 120 defines a bore 202 extending through the fluid permeable body 120 from a first body end 121 of the fluid permeable body 120 to a second body end 123 of the fluid permeable body 120 distal to the first body end 121. In other embodiments, the bore 202 extends only partially into the fluid permeable body from the first body end 121 of the fluid permeable body 120.

In the illustrated embodiment, the conduit 108 is at least partially disposed in the chamber 104 and interfaces at least a portion of the bore 202 of the fluid permeable body 120. For example, the conduit 108 may extend into the fluid impermeable barrier 102 from the first end region 125 (e.g., proximate to the outlet 112) and may extend through the bore 202 to the second end region 127 (e.g., opposite the first end region 125) to a point proximate to a reservoir 122 such that the inlet 110 is in fluid communication with the reservoir 122. For example, in the illustrated embodiment, the inlet 110 is positioned in the reservoir 122. However, in other embodiments such as in FIG. 2B, the inlet 110 may be positioned flush with or behind an end of the fluid permeable body 120 that partially defines the reservoir 122. The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex.

The fluid impermeable barrier 102 can store fluids in a reservoir 122 therein. The reservoir 122 is an unoccupied portion of the chamber 104 and is void of other material. In some embodiments, the reservoir 122 is defined at least partially by the fluid permeable body 120 and the fluid impermeable barrier 102. The reservoir 122 may be disposed in any portion of the interior region of the chamber 104. For example, the fluid reservoir 122 may be positioned in the second end region 127 of the chamber 104. In the illustrated embodiment, the reservoir 122 is defined by the second body end 123 of the fluid permeable body 120 and the second end region 127 of the fluid impermeable barrier 102.

In an embodiment, the fluid impermeable barrier 102 can be air permeable and fluid impermeable. In such an embodiment, the fluid impermeable barrier 102 can be formed of a hydrophobic material that defines a plurality of pores. As described in greater detail above, one or more portions of at least the outer surface 107 of the fluid impermeable barrier 102 can be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

In an embodiment, the fluid permeable body 120 and at least a portion of the conduit 108 can at least substantially completely fill the chamber 104. In another example, the fluid permeable body 120 and at least a portion of the conduit 108 may not substantially completely fill the chamber 104. In such an example, the fluid collection device 100 includes the reservoir 122 disposed in the chamber 104. The reservoir 122 is a substantially unoccupied portion of the chamber 104. The fluids that are in the chamber 104 can flow through the fluid permeable body 120 to the reservoir 122. The reservoir 122 can store at least some of the fluids therein. In these and other embodiments, the fluid permeable body 120, at least a portion of the conduit 108, and the reservoir 122 can at least substantially completely fill the chamber 104.

In an embodiment, the reservoir 122 can be located at the portion of the chamber 104 that is closest to the inlet 110 (e.g., the second end 127 region). However, the reservoir 122 can be located at different locations in the chamber 104 (e.g., the first end 125 region, a portion of the chamber 104 away from the opening 106). In some embodiments, the conduit 108 may extend through the fluid impermeable barrier to the reservoir 122 without extending through the fluid permeable body 120. Accordingly, in these and other embodiments, the fluid permeable body 120 may be free from the bore. In another embodiment, the fluid collection device 100 can include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable body 120 is spaced from at least a portion of the conduit 108 and the reservoir 122 can be the space between the fluid permeable body 120 and the conduit 108.

The fluid impermeable barrier 102, the fluid permeable body 120 can be configured to have the conduit 108 at least partially disposed in the chamber 104. For example, the fluid permeable body 120 can be configured to form a space that accommodates the conduit 108, such as the bore 202. In another example, the fluid impermeable barrier 102 can define an aperture 124 sized to receive the conduit 108 (e.g., at least one tube). The at least one conduit 108 can be disposed in the chamber 104 via the aperture 124. The apertures 124 can be configured to form an at least substantially fluid tight seal against the conduit 108 or the at least one tube thereby substantially preventing the fluids from escaping the chamber 104.

When secured to the fluid collection device 100, the conduit 108 is configured to provide fluid communication with and at least partially extend between one or more of a fluid storage containers (e.g., fluid storage container 458 of FIG. 4) and a portable vacuum source (e.g., vacuum device 470 of FIG. 4). For example, the conduit 108 may be configured to be fluidly coupled to and at least partially extend between one or more of the fluid storage containers and the portable vacuum source. In an embodiment, the conduit 108 is configured to be directly connected to the portable vacuum source. In such an example, the conduit 108 can extend from the fluid impermeable barrier 102 by at least one foot, at least two feet, at least three feet, or at least six feet. In another example, the conduit 108 is configured to be indirectly connected to at least one of the fluid storage container or the portable vacuum source. In some examples, the conduit may be frosted or opaque (e.g., black) to obscure visibility of the fluids therein. In some embodiments, the conduit is secured to a wearer’s skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 are configured to provide fluid communication (e.g., directly or indirectly) between the portable vacuum source (e.g., such as vacuum device 470 in FIG. 4) and the chamber 104 (e.g., the reservoir 122). For example, the inlet 110 and the outlet 112 of the conduit 108 may be configured to directly or indirectly fluidly couple the portable vacuum source to the reservoir 122. In an embodiment, the inlet 110 and/or the outlet 112 can form a male connector. In another example, the inlet 110 and/or the outlet 112 can form a female connector. In an embodiment, the inlet 110 and/or the outlet 112 can include ribs that are configured to facilitate secure couplings. In an embodiment, the inlet 110 and/or the outlet 112 can form a tapered shape. In an embodiment, the inlet 110 and/or the outlet 112 can include a rigid or flexible material.

Locating the inlet 110 at or near a gravimetrically low point of the chamber 104 may enable the conduit 108 to receive more of the fluids than if inlet 110 was located elsewhere and reduce the likelihood of pooling (e.g., pooling of the fluids can cause microbe growth and foul odors). For instance, the fluids in the fluid permeable body 120 can flow in any direction due to capillary forces. However, the fluids may exhibit a preference to flow in the direction of gravity, especially when at least a portion of the fluid permeable body 120 is saturated with the fluids.

As the portable vacuum source applies a vacuum/suction in the conduit 108, the fluid(s) in the chamber 104 (e.g., such as in the reservoir 122 positioned at the first end region 125, the second end region 127, or other intermediary positions within the chamber 104) may be drawn into the inlet 110 and out of the fluid collection device 100 via the conduit 108.

In an embodiment, the conduit 108 is configured to be at least insertable into the chamber 104. In such an embodiment, the conduit 108 can include one or more markers 131 (shown in FIG. 1A) on an exterior thereof that are configured to facilitate insertion of the conduit 108 into the chamber 104. For example, the conduit 108 can include one or more markings thereon that are configured to prevent over or under insertion of the conduit 108, such as when the conduit 108 defines an inlet 110 that is configured to be disposed in or adjacent to the reservoir 122. In another embodiment, the conduit 108 can include one or more markings thereon that are configured to facilitate correct rotation of the conduit 108 relative to the chamber 104. In an embodiment, the one or more markings can include a line, a dot, a sticker, or any other suitable marking. In examples, the conduit 108 may extend into the fluid impermeable barrier 102 from the first end 125 region (e.g., proximate to the outlet 112) and may extend to the second end 127 region (e.g., opposite the first end region) to a point proximate to the reservoir 122 such that the inlet 110 is in fluid communication with the reservoir 122. In some embodiments (not shown), the conduit 108 may enter the second end 127 region and the inlet 110 may be disposed in the second end 127 region (e.g., in the reservoir 122). The fluid collected in the fluid collection device 100 may be removed from the interior region of the chamber 104 via the conduit 108. The conduit 108 may include a flexible material such as plastic tubing (e.g., medical tubing) as disclosed herein. In some examples, the conduit 108 may include one or more portions that are resilient, such as to by having one or more of a diameter or wall thickness that allows the conduit to be flexible.

In an embodiment, one or more components of the fluid collection device 100 can include an antimicrobial material, such as an antibacterial material where the fluid collection device may contact the wearer or the bodily fluid of the wearer. The antimicrobial material can include an antimicrobial coating, such as a nitrofurazone or silver coating. The antimicrobial material can inhibit microbial growth, such as microbial growth due to pooling or stagnation of the fluids. In an embodiment, one or more components of the fluid collection device 100 (e.g., impermeable barrier 102, conduit 108, etc.) can include an odor blocking or absorbing material such as a cyclodextrine containing material or a TPE polymer.

In any of the embodiments disclosed herein the conduits 108 may include or be operably coupled to a flow meter (not shown) to measure the flow of fluids therein, one or more securement devices (e.g., a StatLock securement device, not shown) or fittings to secure the conduit 108 to one or more components of the systems or devices disclosed herein (e.g., portable vacuum source or fluid storage container as described in more detail with reference to FIG. 4), or one or more valves to control the flow of fluids in the systems and devices herein.

In an embodiment, at least one of portion of the conduit 108 of the fluid collection devices or systems herein can be formed of an at least partially opaque material which can obscure the fluids that are present therein. For example, the section of the conduits 108 disclosed herein may be formed of an opaque material or translucent material while the A section may be formed of a transparent material or translucent material. In some embodiments, the B section may include transparent or translucent material. Unlike the opaque or nearly opaque material, the translucent material allows a user of the devices and systems herein to visually identify fluids or issues that are inhibiting the flow of fluids within the conduit 108.

Fluid collection devices described herein may be used in fluid collection systems. The fluid collection systems can include a fluid collection device, a fluid storage container, and a portable vacuum source. Fluid (e.g., urine or other bodily fluids) collected in the fluid collection device may be removed from the fluid collection device via a conduit which protrudes into an interior region of the fluid collection device. For example, a first open end of the conduit may extend into the fluid collection device to a reservoir therein. The second open end of the conduit may extend into the fluid collection device or the portable vacuum source. The suction force may be introduced into the interior region of the fluid collection device via the first open end of the conduit responsive to a suction (e.g., vacuum) force applied at the second end of the conduit. The suction force may be applied to the second open end of the conduit by the portable vacuum source either directly or indirectly.

In some embodiments, the portable vacuum source may be disposed in or on the fluid collection device. In such embodiments, the conduit may extend from the fluid collection device and attach to the portable vacuum source at a first point therein. An additional conduit may attach to the portable vacuum source at a second point thereon and may extend out of the fluid collection device, and may attach to the fluid storage container. Accordingly, a vacuum (e.g., suction) may be drawn through fluid collection device via the fluid storage container. Fluid, such as urine, may be drained from the fluid collection device using the portable vacuum source.

FIG. 4 is a schematic illustration of system 456 that includes a fluid collection device 400 according to at least one embodiment of the disclosure. The fluid collection device 400 may include any of the fluid collection assemblies disclosed herein, such as fluid collection device 400. The fluid collection device 400 may be in fluid communication with a fluid storage container 458 via at least one first conduit 408 (e.g., conduit 108, 608 of FIGS. 1-2). The fluid storage container 458 is positioned downstream from the fluid collection device 400. The fluid storage container 458 may be in fluid communication with a vacuum device 470 via at least one second tube 462. The vacuum device 470 is positioned downstream from the fluid storage container 458. During operation, the vacuum device 470 provides a suction force to the fluid collection device 400. The suction force draws fluid into the chamber and towards the first conduit 408. The fluid that enters the first conduit 408 is pulled by the suction force towards the fluid storage container 458 such that the fluid storage container 458 receives the fluid. The fluid storage container 458 may be configured to inhibit the fluid from flowing from the fluid storage container 458 to the vacuum device 470.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

Additional embodiments of a fluid permeable body will now be described with reference to FIGS. 5-13. The embodiments of the fluid permeable body disclosed herein may be used to implement and/or replace the fluid permeable body 120 of the fluid collection device 100 shown in FIGS. 1-3.

FIG. 5 is a cross-sectional view along a long axis of a fluid collection device 500 including a fluid impermeable barrier and a fluid permeable body according to at least one embodiment of the disclosure. In the example shown in FIG. 5, the fluid impermeable barrier 502 may be substantially the same as the fluid impermeable barrier 102, and the conduit 508 may be substantially the same as the conduit 108. Accordingly, the details of fluid impermeable barrier 502 and conduit 508 will not be discussed.

The fluid permeable body 520 may include a fluid permeable membrane 540 at least partially surrounding a fluid permeable support 542, similar to fluid permeable membrane 340 and fluid permeable support 342. In some embodiments, the fluid permeable support 542 may be substantially similar to fluid permeable support 342. In some embodiments, in contrast to fluid permeable membrane 340, the fluid permeable membrane 540 may include a hydrophilic material. The entire fluid permeable membrane 540 may include the hydrophilic material in some embodiments or may include a hydrophilic coating on a hydrophobic and/or neutral material. Examples of suitable materials may include a microfiber, nonwoven polypropylene, and/or nonwoven polyethelene.

In some applications, a hydrophilic fluid permeable membrane 540 may improve performance of the fluid permeable body 520 when initially dry. In some applications, the hydrophilic fluid permeable membrane 540 may allow bodily fluids to spread more quickly along a length of the fluid permeable body 520. This may improve movement of fluid through the fluid permeable support 542, removal of fluid from the fluid collection device 500, reduce a risk of leaks, and/or reduce feelings of wetness by a wearer.

In some embodiments, the hydrophilic fluid permeable membrane 540 may include tapered pores. The pores may be tapered such that the pores are wider on an outer surface 521 of the fluid permeable membrane 540 than on an inner surface 523 of the fluid permeable membrane 540. This may make it easier for fluid to pass from the outer surface 521 to the inner surface 523 and more difficult for fluid to pass from the inner surface 523 to the outer surface 521. In some embodiments, the hydrophilic fluid permeable membrane 540 may include a hydrophobic coating on the inner surface 523. This may make the energy barrier higher at the inner surface 523 than the outer surface 521. That is, it may be easier for fluid to pass from the outer surface 521 to the inner surface 523 than from the inner surface 523 to the outer surface 521. In some embodiments, the hydrophilic fluid permeable membrane 540 may include both tapered pores and a hydrophobic coating on the inner surface 523.

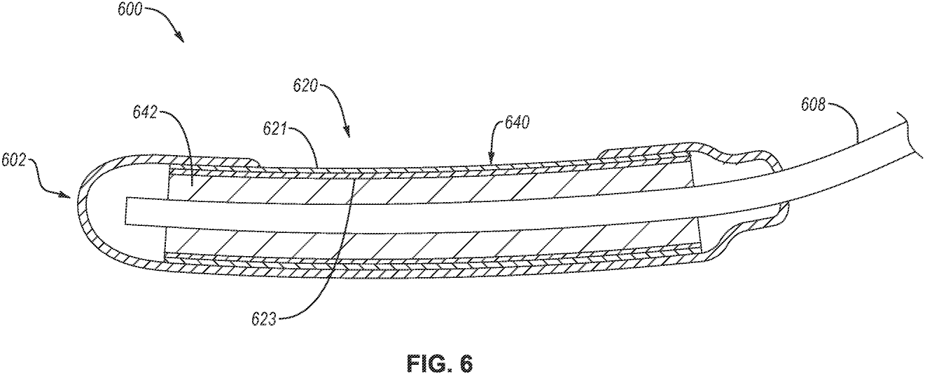

FIG. 6 is a cross-sectional view along a long axis of a fluid collection device 600 including a fluid impermeable barrier and a fluid permeable body according to at least one embodiment of the disclosure. In the example shown in FIG. 6, the fluid impermeable barrier 602 may be substantially the same as the fluid impermeable barrier 102, and the conduit 608 may be substantially the same as the conduit 108. Accordingly, the details of fluid impermeable barrier 602 and conduit 608 will not be discussed.

The fluid permeable body 620 may include a fluid permeable membrane 640 at least partially surrounding a fluid permeable support 642, similar to fluid permeable membrane 340 and fluid permeable support 342. In some embodiments, the fluid permeable support 642 may be substantially similar to fluid permeable support 342. In the embodiment shown in FIG. 6, the fluid permeable membrane 640 may include a wicking fabric. The wicking fabric of the fluid permeable membrane 640 may be different than other wicking fabrics that wick fluids from a surface, such as outer surface 621 and then merely distribute the fluids within the fabric. This may leave a wearer of the fluid collection device 600 feeling wet, reducing comfort. Instead, the wicking fabric of the fluid permeable membrane 640 may wick fluids away from an outer surface 621 of the fluid permeable membrane 640 to an inner surface 623. In some embodiments, fluid wicked to the inner surface 623 may be transmitted to and/or through the fluid permeable support 642.

An example of a suitable fabric that may be used for fluid permeable membrane 640 is “Dry Inside” fabric available from NANOtex®, a Crypton Company headquartered in Bloomfield Hills, Michigan. The Dry Inside fabric may wick fluids. In addition to providing wicking functions, the Dry Inside fabric may provide comfort (e.g., less chafing, soft feeling) to a wearer of the fluid collection device.

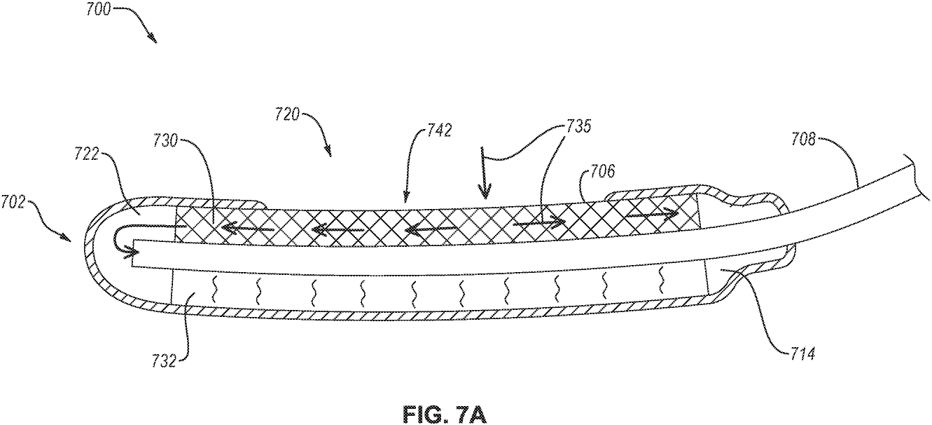

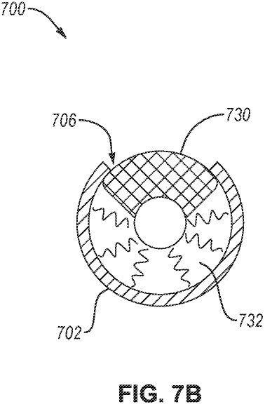

FIG. 7A is a cross-sectional view along a long axis of a fluid collection device 700 including a fluid impermeable barrier and a fluid permeable body according to at least one embodiment of the disclosure. In the example shown in FIG. 7A, the fluid impermeable barrier 702 may be substantially the same as the fluid impermeable barrier 102, and the conduit 708 may be substantially the same as the conduit 108. Accordingly, the details of fluid impermeable barrier 702 and conduit 708 will not be discussed.

The fluid permeable body 720 may include a fluid permeable support 742. The fluid permeable support 742 may include two portions: a wicking portion 730 and a packing portion 732. The fluid permeable body 720 may be positioned within a chamber 704 of the fluid impermeable barrier 702 such that at least a portion of the wicking portion 730 is exposed by an opening 706 defined by the fluid impermeable barrier 702 and the packing portion 732 is positioned within the chamber 704 such that all or a majority of the packing portion 732 is not exposed by the opening 706.

In some embodiments, the wicking portion 730 may include a fluid permeable material such as an open cell foam, a nonwoven material, an open capillary foam, and/or a filter medium. Open cell foams may include one or more materials. Examples of open cell foam materials include, but are not limited to polyurethane, open cell rubber, polyester, polyamide. In some embodiments, the packing portion 732 may include a non-absorbent material impervious to air such as a closed cell foam. Closed cell foams may include one or more materials. Examples of closed cell foam materials include, but are not limited to, ethylene propylene diene monomer (EPDM), neoprene, polyethylene, polystyrene, polypropylene, and ethylene-vinyl acetate (EVA). Of course, some materials, such as polyurethane and polypropylene, for example, may be used to make either closed cell or open cell foam.

As indicated by arrows 735, fluid may contact the wicking portion 730 and be drawn along the length of the wicking portion 730 to a reservoir 722 where it may be collected and removed from the fluid collection device 700 via the conduit 708. The fluid may be drawn by capillary action, gravity, and/or suction. By only wicking fluid through a portion of the fluid permeable body 720, the flow path of fluid and/or air may be condensed. This may keep fluid together which may allow the fluid to drain better due to reduced surface area between the fluid permeable body 720 and the fluid. The condensed flow path may also allow suction from a vacuum source (e.g., vacuum device 470) to be concentrated, which may improve removal of fluid from the fluid collection device.

FIG. 7B is a cross-sectional view along a short axis of the fluid collection device 700 including the fluid impermeable barrier 702 and the fluid permeable body 720. As shown in FIG. 7B, the wicking portion 730 may extend such that a portion of the wicking portion 730 is not exposed by the opening 706 of the fluid impermeable barrier 702. However, in other embodiments, the wicking portion 730 may extend to be substantially aligned with the edges of the opening 706. In other embodiments, the wicking portion 730 may not span the entire opening 706 such that some of the packing portion 732 is exposed by the opening 706.

As shown in FIG. 7B, the wicking portion 730 of the fluid permeable body 720 may be approximately one third and the packing portion 732 may be approximately two thirds of the circumference of the fluid permeable body 720. Other ratios may be used in other embodiments. For example, the wicking portion 730 may be approximately one half and the packing portion 732 may be approximately one half of the circumference of the fluid permeable body 720. In another example, the wicking portion 730 may be approximately one quarter and the packing portion 732 may be approximately three quarters of the circumference of the fluid permeable body 720. In some embodiments, the ratio of the wicking portion 730 to the packing portion 732 may be based, at least in part, on a size of the opening 706. In some embodiments, the ratio of the wicking portion 730 to the packing portion 732 may be based, at least in part, on a desired size of the flow path for fluid and/or air.

In some embodiments, the wicking portion 730 may include a material smooth and/or soft enough to be suitable for contact with a wearer’s skin, and the fluid permeable body may not include a fluid permeable membrane over the fluid permeable support 742. In other embodiments, the fluid permeable support 742 may be at least partially covered by a fluid permeable membrane (not shown), such as a fluid permeable membranes disclosed herein (e.g., fluid permeable membrane 340, 540, and/or 640).

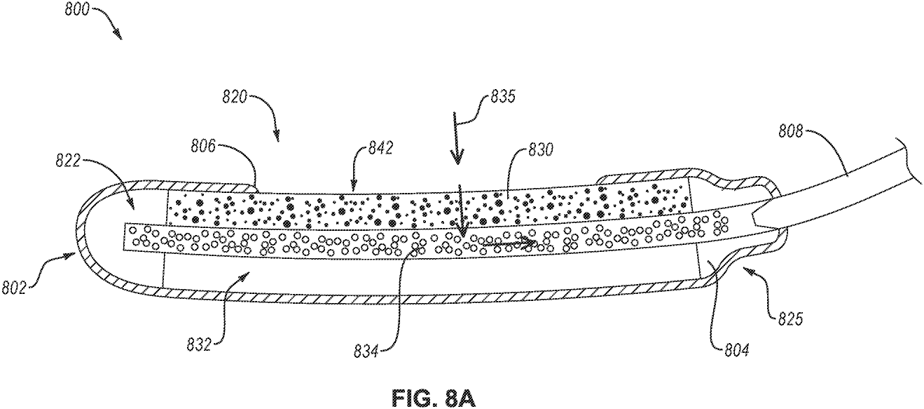

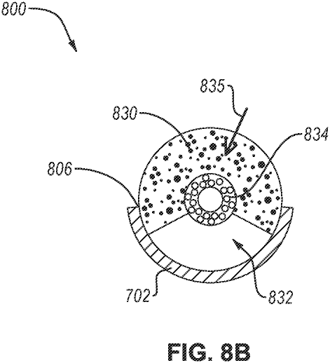

FIG. 8A is a cross-sectional view along a long axis of a fluid collection device 800 including a fluid impermeable barrier and a fluid permeable body according to at least one embodiment of the disclosure. In the example shown in FIG. 8A, the fluid impermeable barrier 802 may be substantially the same as the fluid impermeable barrier 102. Accordingly, the details of fluid impermeable barrier 802 will not be discussed.

The fluid collection device 800 may include a fluid permeable body 820 positioned at least partially within the fluid impermeable barrier 802. The fluid permeable body 820 may include a fluid permeable support 842. The fluid permeable support 842 may include an inverse sparger system. The inverse sparger system may include a wicking portion 830, a packing portion 832, and a sparger tube 834. In some embodiments, such as the one shown in FIG. 8A, a conduit 808 coupled to the fluid impermeable barrier 802 may be coupled to the sparger tube 834 near a first end portion of the fluid impermeable barrier 802. The fluid permeable body 820 may be positioned within a chamber 804 of the fluid impermeable barrier 802 such that at least a portion of the wicking portion 830 is exposed by an opening 806 defined by the fluid impermeable barrier 802 and the packing portion 832 is positioned within the chamber 804 such that all or a majority of the packing portion 832 is not exposed by the opening 806.

In some embodiments, the wicking portion 830 may include an open cell foam. In some embodiments, the packing portion 832 may also include an open cell foam, but in other embodiments, the packing portion 832 may include a closed cell foam or other material impervious to fluid and/or air. The sparger tube 834 may include small holes (e.g., pores, openings) along a length of the tube 834. In some embodiments, the openings of the wicking portion 830 may be larger than the holes of the sparger tube 834. In some embodiments, the sparger tube 834 may include a porous metal tube. In some embodiments, the sparger tube 834 may include a porous polymer tube. In some embodiments, such as the one shown in FIG. 8A, the sparger tube 834 may extend beyond the wicking portion 830 and packing portion 832 at either end of the fluid permeable body 820. However, in other embodiments, the sparger tube 834 may extend to be substantially equal with one or both ends of the wicking portion 830 and packing portion 832.

As indicated by arrows 835, fluid may enter the wicking portion 830 and drawn into the sparger tube 834 where the fluid is then drawn toward the conduit 808 for removal from the fluid collection device 800. The fluid may be drawn by capillary action, gravity, and/or suction. The sparger tube 834 may allow for equilibrium of pressure along a length of the sparger tube 834. This may allow fluids to be collected along a greater length of the tube rather than having to be transmitted to a reservoir 822 prior to being drawn into the conduit 808.

FIG. 8B is a cross-sectional view along a short axis of the fluid collection device 800 including the fluid impermeable barrier 802 and the fluid permeable body 820. As shown in FIG. 8B, the wicking portion 830 may extend such that a portion of the wicking portion 830 is not exposed by the opening 806 of the fluid impermeable barrier 802. However, in other embodiments, the wicking portion 830 may extend to be substantially aligned with the edges of the opening 806. In other embodiments, the wicking portion 830 may not span the entire opening 806 such that some of the packing portion 832 is exposed by the opening 806.

As shown in FIG. 8B, the wicking portion 830 of the fluid permeable body 820 may be approximately two thirds and the packing portion 832 may be approximately one third of the circumference of the fluid permeable body 820. Other ratios may be used in other embodiments. For example, the wicking portion 830 may be approximately one half and the packing portion 832 may be approximately one half of the circumference of the fluid permeable body 820. In another example, the wicking portion 830 may be approximately one quarter and the packing portion 832 may be approximately three quarters of the circumference of the fluid permeable body 820. In a further example, the wicking portion 830 may be approximately three quarters and the packing portion 832 may be approximately one quarter of the circumference of the fluid permeable body 820. In some embodiments, the ratio of the wicking portion 830 to the packing portion 832 may be based, at least in part, on a size of the opening 806. In some embodiments, the ratio of the wicking portion 830 to the packing portion 832 may be based, at least in part, on a desired equilibrium pressure of fluid and/or air along the sparger tube 834.

In some embodiments, the wicking portion 830 may include a material smooth and/or soft enough to be suitable for contact with a wearer’s skin, and the fluid permeable body may not include a fluid permeable membrane over the fluid permeable support 842. In other embodiments, the fluid permeable support 842 may be at least partially covered by a fluid permeable membrane (not shown), such as a fluid permeable membranes disclosed herein (e.g., fluid permeable membrane 340, 540, and/or 640).

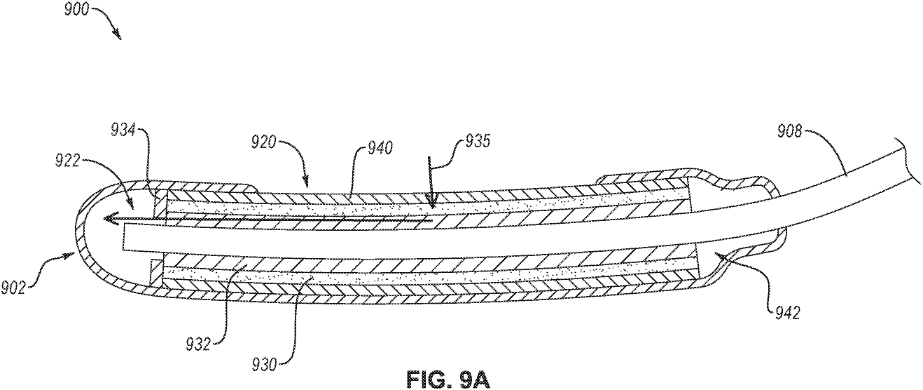

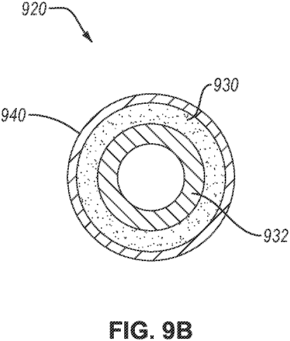

FIG. 9A is a cross-sectional view along a long axis of a fluid collection device 900 including a fluid impermeable barrier and a fluid permeable body according to at least one embodiment of the disclosure. In the example shown in FIG. 9A, the fluid impermeable barrier 902 may be substantially the same as the fluid impermeable barrier 102, and the conduit 908 may be substantially the same as the conduit 108. Accordingly, the details of fluid impermeable barrier 902 and conduit 908 will not be discussed.

The fluid permeable body 920 may include a fluid permeable membrane 940 at least partially surrounding a fluid permeable support 942, similar to fluid permeable membrane 340 and fluid permeable support 342. In some embodiments, the fluid permeable membrane 940 may include fluid permeable membrane 340, 540, 640, and/or any other fluid permeable membrane disclosed herein.

In some embodiments, such as the one shown in FIG. 9A, the fluid permeable support 942 may include two or more concentric layers: an outer layer 930 adjacent to the fluid permeable membrane 940 and an inner layer 932 adjacent to the conduit 908. The concentricity of the layers is also visible in FIG. 9B which shows a cross-sectional view along a short axis of the fluid permeable body 920 of the fluid collection device 900. Although two layers are shown in the embodiment in FIGS. 9A-9B, more than two layers may be included in different embodiments. In some embodiments, the porosity may decrease and/or density may increase with each layer from the innermost to the outer most layer. In other embodiments, the porosity may increase and/or density may decrease with each layer from the innermost to the outermost layer.

In some embodiments, the outer layer 930 and the inner layer 932 may have different densities and/or porosities. In some embodiments, the outer layer 930 may have a higher density/lower porosity than the inner layer 932. In other embodiments, the inner layer 932 may have a higher density/lower porosity than the outer layer 930. In some embodiments, the outer layer 930 and inner layer 932 may be different density open cell foams. In some embodiments, the lower density/higher porosity layer may include a reticulated foam. The two layers may have the same or different material and/or chemical compositions. In some embodiments, the layers may include polyester, polypropylene, and/or polyurethane.

As shown by arrows 935, fluid may be wicked through the fluid permeable membrane 940 and the outer layer 930 of the fluid permeable support 942. The fluid may further be drawn to the inner layer 932 and toward a reservoir 922 of the fluid collection device 900 where the fluid may be received and removed from the fluid collection device 900 by conduit 908. The fluid may be drawn by capillary action, gravity, and/or suction (e.g., as provided by a vacuum device, such as vacuum device 470).

In some embodiments, such as the one shown in FIG. 9A, the fluid permeable body 920 may include a fluid impermeable cap 934 on at least one end of the fluid permeable body 920. The cap 934 may have an annular shape that covers at least an edge of the fluid permeable membrane 940 and an edge of the outer layer 930 of the fluid permeable support 942. The cap 934 may leave at least a portion of the edge of the inner layer 932 uncovered. The cap 934 may encourage fluid and/or air flow through the inner layer 932. In some embodiments, cap 934 may instead be an annular-shaped extension from an inner surface of the fluid impermeable barrier 902. In these embodiments, the cap 934 may further act as a stop to help position the fluid permeable body 920 within the fluid impermeable barrier 902.

In some embodiments, the high density/low porosity of the outer layer 930 or inner layer 932 may equilibrate pressure in the low density/high porosity of the inner layer 932 or outer layer 930 along a length of the fluid permeable body 920 due to the higher air flow resistance in the high density/low porosity material. This may permit better fluid wicking along a length of the fluid permeable body 920 and/or make it easier for light fluid flows to break the surface tension of the fluid permeable membrane 940 and/or outer layer 930. In some applications, the improved wicking and/or reduced surface tension may reduce dependence on anatomical fit and/or placement for proper performance of the fluid collection device 900.

Although the fluid permeable body 920 is shown including the fluid permeable membrane 940, in embodiments where the outer layer 930 of the fluid permeable support 942 is sufficiently comfortable against a wearer’s skin, the fluid permeable membrane 940 may be omitted.

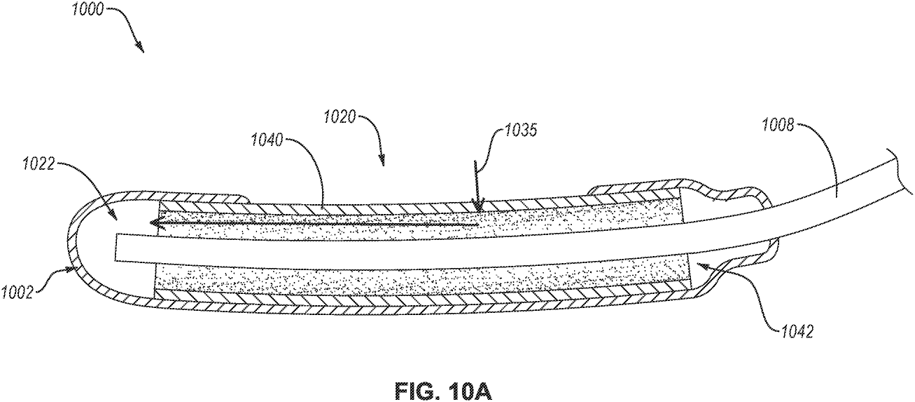

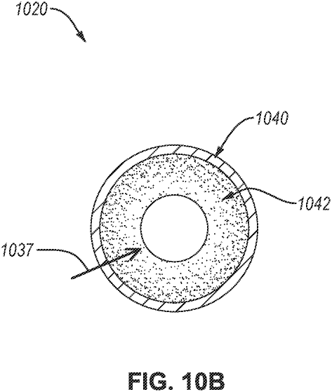

FIG. 10A is a cross-sectional view along a long axis of a fluid collection device 1000 including a fluid impermeable barrier and a fluid permeable body according to at least one embodiment of the disclosure. In the example shown in FIG. 10A, the fluid impermeable barrier 1002 may be substantially the same as the fluid impermeable barrier 102, and the conduit 1008 may be substantially the same as the conduit 108. Accordingly, the details of fluid impermeable barrier 1002 and conduit 1008 will not be discussed.

The fluid permeable body 1020 may include a fluid permeable membrane 1040 at least partially surrounding a fluid permeable support 1042, similar to fluid permeable membrane 340 and fluid permeable support 342. In some embodiments, the fluid permeable membrane 1040 may include fluid permeable membrane 340, 540, 640, and/or any other fluid permeable membrane disclosed herein.

In some embodiments, rather than including multiple layers as shown in FIGS. 9A-9B, the fluid permeable support 1042 may include a single material having a graded porosity and/or density. In some embodiments, the porosity and/or density may change along the fluid permeable support 1042 in a direction extending between the conduit 1008 and the fluid permeable membrane 1040. The direction of the gradient of the density and/or porosity is shown by arrow 1037 in FIG. 10B which shows a cross-sectional view along a short axis of the fluid permeable body 1020 of the fluid collection device 1000. In some embodiments, the porosity may be less and the density may be greater near the conduit 1008 compared to near the fluid permeable membrane 1040. In other embodiments, the porosity may be less and the density may be greater near the fluid permeable membrane 1040 compared to near the conduit 1008. In some embodiments, the fluid permeable support 1042 may include a gradient density and/or gradient porosity foam. In some embodiments, the fluid permeable support 1042 may include polyester, polypropylene, and/or polyurethane.

As shown by arrows 1035, fluid may be wicked through the fluid permeable membrane 1040 and through the gradient fluid permeable support 1042 to an inner portion near the conduit 1008. The fluid may further be drawn through the inner portion toward a reservoir 1022 of the fluid collection device 1000 where the fluid may be received and removed from the fluid collection device 1000 by conduit 1008. The fluid may be drawn by capillary action, gravity, and/or suction (e.g., as provided by a vacuum device, such as vacuum device 470).

In some embodiments, similar to the embodiment described with reference to FIGS. 9A-9B, the high density/low porosity portion of the gradient fluid permeable support 1042 may equilibrate pressure in the low density/high porosity portion of the fluid permeable support 1042 along a length of the fluid permeable body 1020 due to the higher air flow resistance in the high density/low porosity material. This may permit better fluid wicking along a length of the fluid permeable body 1020 and/or make it easier for light fluid flows to break the surface tension of the fluid permeable membrane 1040 and/or fluid permeable support 1042. In some applications, the improved wicking and/or reduced surface tension may reduce dependence on anatomical fit and/or placement for proper performance of the fluid collection device 1000.

Although the fluid permeable body 1020 is shown including the fluid permeable membrane 1040, in embodiments where an outer surface of the fluid permeable support 1042 is sufficiently comfortable against a wearer’s skin, the fluid permeable membrane 1040 may be omitted.

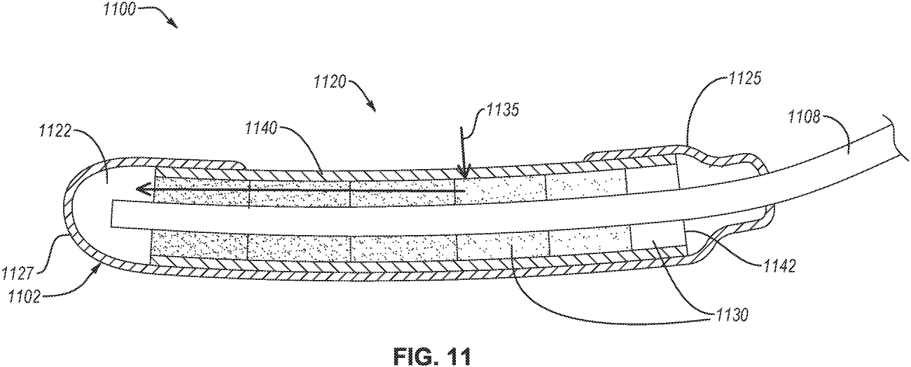

FIG. 11 is a cross-sectional view along a long axis of a fluid collection device 1100 including a fluid impermeable barrier and a fluid permeable body according to at least one embodiment of the disclosure. In the example shown in FIG. 11, the fluid impermeable barrier 1102 may be substantially the same as the fluid impermeable barrier 102, and the conduit 1108 may be substantially the same as the conduit 108. Accordingly, the details of fluid impermeable barrier 1102 and conduit 1108 will not be discussed.

The fluid permeable body 1120 may include a fluid permeable membrane 1140 at least partially surrounding a fluid permeable support 1142, similar to fluid permeable membrane 340 and fluid permeable support 342. In some embodiments, the fluid permeable membrane 1140 may include fluid permeable membrane 340, 540, 640, and/or any other fluid permeable membrane disclosed herein.

In some embodiments, the fluid permeable support 1142 may include one or more annular portions 1130 extending along a portion of the length of the fluid permeable body 1120. Each annular portion 1130 may have a different density and/or porosity. In some embodiments, the porosity may decrease and/or the density may increase for each annular portion 1130 from a first end 1125 of the fluid collection device 1100 to a second end 1127 of the fluid collection device 1100. In other words, an annular portion 1130 proximate the first end 1125 may have a higher porosity and/or lower density than an annular portion 1130 proximate the second end 1127. Alternatively, in some embodiments, the fluid permeable support 1142 includes a material with a gradient porosity and/or density such that the porosity is greatest and density is lowest at the first end 1125 and the porosity is lowest and density is greatest at the second end 1127.

In some embodiments, the fluid permeable support 1142 may include one or more open cell foams. In some embodiments, the fluid permeable support 1142 may include polyester, polypropylene, and/or polyurethane foams.

As shown by arrows 1135, fluid may be wicked through the fluid permeable membrane 1140 and through a length of the fluid permeable support 1142 through decreasing porosity and/or increasing density material toward a reservoir 1122 of the fluid collection device 1100 where the fluid may be received and removed from the fluid collection device 1100 by conduit 1108. The fluid may be drawn by capillary action, gravity, and/or suction (e.g., as provided by a vacuum device, such as vacuum device 470).

In some applications, the first end 1125 of the fluid collection device 1100 may be closer to a urethral opening of a wearer than the second end 1127. Accordingly, the fluid permeable body 1120 proximate the first end 1125 may be more likely to experience bolus flow in these applications. The lower density and/or higher porosity of the fluid permeable support 1142 in this region may provide greater capacity for managing bolus flow (e.g., less likely to experience runoff and/or over saturation) and drawing the fluid into the fluid collection device 1100 and away from the wearer. The higher density and/or lower porosity portion of the fluid permeable support 1142 proximate the second end 1127 may condense the flow path of fluid and/or air, which may encourage fluid flow to the reservoir 1022 where the fluid can be removed from the fluid collection device 1100 by the conduit 1108.

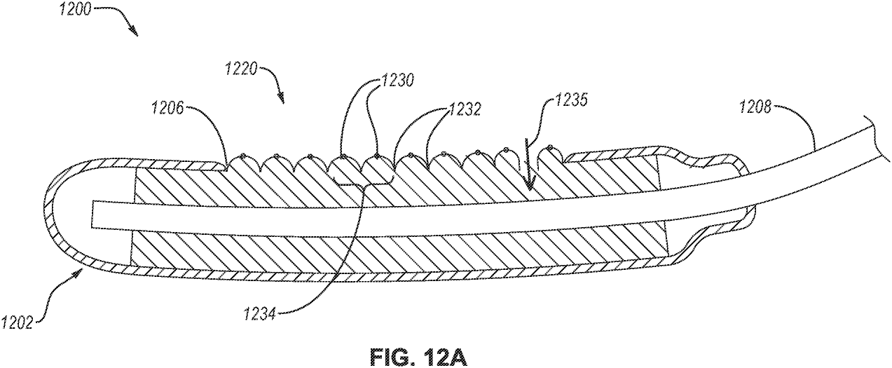

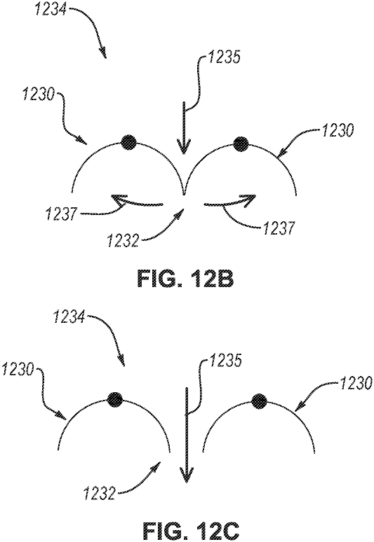

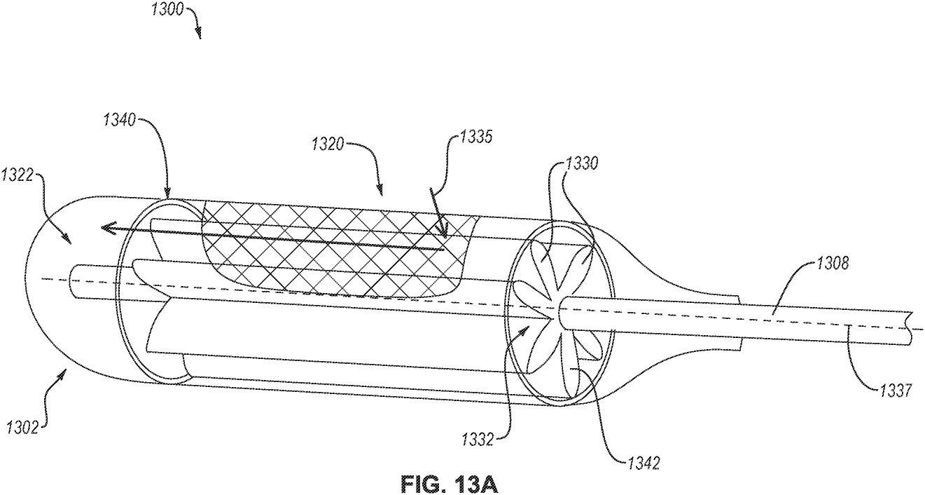

FIG. 12A is a cross-sectional view along a long axis of a fluid collection device 1200 including a fluid impermeable barrier and a fluid permeable body according to at least one embodiment of the disclosure. In the example shown in FIG. 12A, the fluid impermeable barrier 1202 may be substantially the same as the fluid impermeable barrier 102, and the conduit 1208 may be substantially the same as the conduit 108. Accordingly, the details of fluid impermeable barrier 1202 and conduit 1208 will not be discussed.

The fluid permeable body 1220 may include a fluid permeable membrane 1240 at least partially surrounding a fluid permeable support (not shown), similar to fluid permeable membrane 340 and fluid permeable support 342. In some embodiments, the fluid permeable support may be substantially similar to fluid permeable support 342. However, other fluid permeable supports may be used, for example, fluid permeable support 542, 642, 742, 842, 942, 1042, and/or 1142.

In some embodiments, the fluid permeable membrane 1240 may include multiple folds 1230 (e.g., bulges, ridges) in the material forming the fluid permeable membrane 1240. In between the folds 1230 may be slits 1232. The slits 1232 may pass all the way through the fluid permeable membrane 1240. Two folds 1230 on either side of a slit 1232 may form a slot valve 1234. Slot valves may also be referred to as duckbill valves or sprung valves. In some embodiments, the fluid permeable membrane 1240 may include a nonwoven material. In some embodiments, the fluid permeable membrane 1240 may include polypropylene or other suitable polymer. In some embodiments, natural fibers may be included in the fluid permeable membrane 1240.

In some embodiments, the entire fluid permeable membrane 1240 includes the slot valves 1234 formed by the folds 1230 and slits 1232. In other embodiments, only a portion of the fluid permeable membrane 1240 includes the slot valves 1234. For example, in some embodiments, only the portion of the fluid permeable membrane 1240 exposed by an opening 1206 in the fluid impermeable barrier 1202 includes the slot valves 1234.

FIGS. 12B and 12C illustrate the operation of the slot valve 1234 in more detail. FIG. 12B illustrates a slot valve 1234 in a “closed” position. As shown in FIG. 12B, even in the closed position, the slot valve 1234 is still slightly open, which may permit fluid to pass through the slot valve 1234, and thus pass through the fluid permeable membrane 1240. This may permit even stagnant fluids (e.g., slow urine leaks) to be wicked into the fluid collection device 1200. As indicated by arrow 1235, a fluid flow entering between two folds 1230 may apply a force indicated by arrows 1237 against the folds 1230. As shown in FIG. 12C, the force from the fluid flow may push apart the folds 1230 and open the slit 1232 wider. Thus, in FIG. 12C, the slot valve 1234 is shown in an open state due to the fluid flow. The greater opening of the slits 1232 may permit the fluid permeable membrane 1240 to accommodate a bolus flow of fluid. In some embodiments, a vacuum within the fluid permeable body 1220, for example, caused by suction provided by a vacuum device (e.g., vacuum device 470) via the conduit 1208 may assist in keeping the slot valves 1234 in the “closed” state as shown in FIG. 12B in the absence of fluid flow.

In some embodiments, the slot valves 1234 may be one-way. That is, fluid flowing in a direction opposite arrow 1235 may not cause the slit 1232 to open further. The one-way property of the slot valves 1234 may help retain fluids within the fluid collection device 1200 and/or prevent leaks.

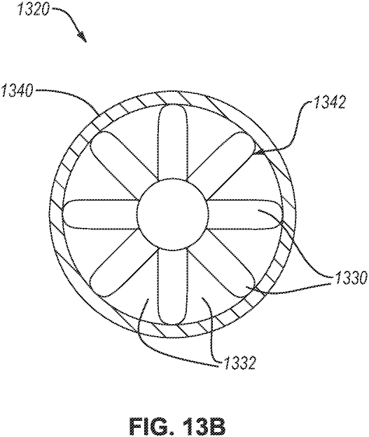

FIG. 13A is a view of a fluid collection device 1300 including a fluid impermeable barrier and a fluid permeable body according to at least one embodiment of the disclosure. In the example shown in FIG. 13A, the fluid impermeable barrier 1302 may be substantially the same as the fluid impermeable barrier 102, and the conduit 1308 may be substantially the same as the conduit 108. Accordingly, the details of fluid impermeable barrier 1302 and conduit 1308 will not be discussed. The fluid permeable body 1320 may include a fluid permeable membrane 1340 at least partially surrounding a support 1342. In some embodiments, the fluid permeable membrane 1040 may include fluid permeable membrane 340, 540, 640, 1240, and/or any other fluid permeable membrane disclosed herein.

The support 1342 may include one or more spokes 1330 extending outward from a central axis 1337 of the fluid permeable body 1320 to the fluid permeable membrane 1340 as can be seen in FIG. 13B, which is a cross-sectional view of the fluid permeable body along a short axis of the fluid collection device 1300. As shown in FIG. 13A, the spokes 1330 may extend along a length of the fluid permeable body 1320, giving the support 1342 the appearance of a paddlewheel. The spokes 1330 may at least partially define spaces 1332 between the spokes 1330. In some embodiments, the spokes 1330 may be formed from a material that is not permeable to air and/or fluid. In some embodiments, the spokes 1330 may include a closed cell foam, an impermeable plastic shell, and/or solid plastic. In some embodiments, the spokes 1330 may include an outer coating and/or material that has wicking properties whereas the interior of the spokes 1330 is impermeable to air and/or fluid.

The impermeability of the spokes 1330 may concentrate airflow (e.g., suction) and/or fluid flow to the spaces 1332. As shown by arrows 1335, fluid may pass through the fluid permeable membrane 1340, along the spaces 1332 to a reservoir 1322 where the fluid may be removed from the fluid collection device via conduit 1308. In some embodiments, concentrating the airflow and/or fluid flow may equilibrate pressure along a length of the fluid permeable body 1320. In some embodiments, concentrating the airflow and/or fluid flow may permit more focused (e.g., stronger) suction. In some embodiments, the spokes 1330 may encourage fluids to remain concentrated (e.g., not spread out through the fluid permeable body 1320), which may improve fluid flow towards the reservoir 1322 and/or into the conduit 1308.

Some or all of the features of the various embodiments of the fluid permeable body shown in FIGS. 5-13 may be used in combination. For example, the hydrophilic fluid permeable membrane of FIG. 5 may be used to form the slot valves of the fluid permeable membrane in FIG. 12. In another example, the wicking portion of the fluid permeable support of FIG. 7 may have a gradient porosity and/or density similar to the fluid permeable support of FIGS. 10A-10B. Any of the embodiments of the fluid permeable body shown in FIGS. 5-13 and/or combinations thereof may be used with the fluid collection device 100 in some embodiments. For example, the fluid permeable bodies shown in FIGS. 5-13 may be at least partially enclosed by the fluid impermeable barrier 102.

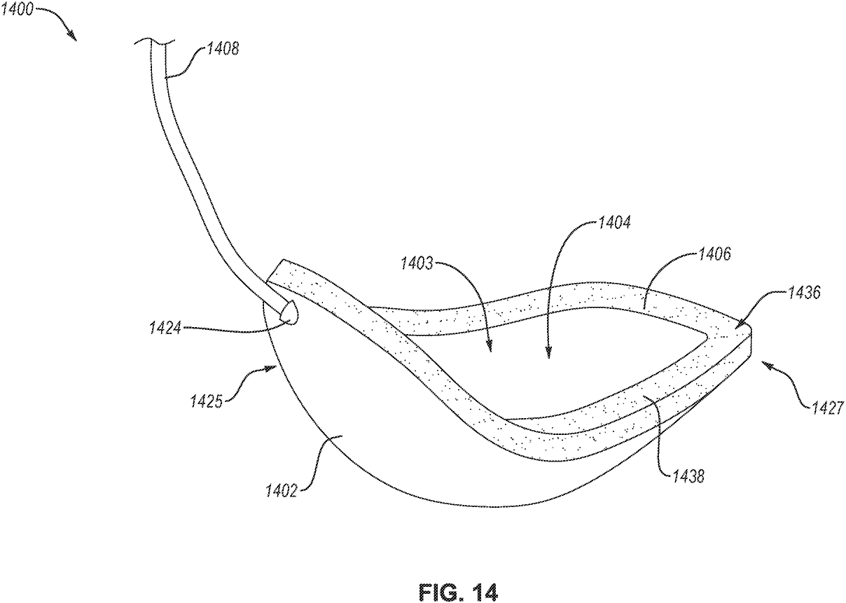

Additional embodiments of a fluid impermeable membrane will now be described with reference to FIG. 14.

FIG. 14 is a view of a fluid collection device 1400 according to at least one embodiment of the present disclosure. In some applications, the fluid collection device 1400 may be desirable for collecting fluids from wearers who are mobile and/or semi-mobile. In some embodiments, the fluid collection device 1400 may include a fluid impermeable barrier 1402. In some embodiments, the fluid impermeable barrier 1402 may include silicone, closed cell foam, poly(p-phenylene ether), and/or any other materials described as included in fluid impermeable barrier 102 of FIGS. 1-3. The fluid impermeable barrier 1402 may at least partially define a chamber 1404 and an opening 1406 to the chamber 1404. Similar to the fluid impermeable barrier 102, the fluid impermeable barrier 1402 may accept fluid into the chamber 1404 through opening 1406. The fluid impermeable barrier 1402 may further define an aperture 1424 at a first end 1425. The aperture 1424 may pass through the fluid impermeable barrier 1402 into the chamber 1404. The aperture 1424 may be sized to accept a conduit 1408. Similar to conduit 108, the conduit 1408 may receive fluids from the chamber 1404 and remove the fluids from the chamber 1404. In some embodiments, the conduit 1408 may provide suction to the chamber 1404, which may facilitate removal of the fluids. In some embodiments, the suction may be provided by a vacuum device, such as vacuum device 470, coupled to the conduit 1408. In some embodiments, the conduit 1408 may extend at least partially into the chamber 1404. In some embodiments, the conduit 1408 may extend through the chamber 1404 at or near a second end 1427 of the fluid impermeable barrier 1402.

In contrast to the generally cylindrical fluid impermeable barrier 102, the fluid impermeable barrier 1402 may have a curved ovular shape. An inner surface 103 of the fluid impermeable barrier 1402 may be generally concave. A perimeter of the opening 1406 may define two opposing generally U-type shaped portions coupled at an angle. The fluid impermeable barrier 1402 may be curved such that from a side view along a long axis of the fluid collection device 1400, the fluid impermeable barrier 1402 approximates an “L” or “J” shape. The fluid impermeable barrier 1402 may generally be shaped to curve around a female wearer’s crotch and/or pubic area. For example, in some embodiments, the first end 1425 may be proximate the wearer’s pubic bone whereas the second end 1427 is proximate the wearer’s perineum. In some embodiments, such as when the conduit 1408 does not extend into the chamber 1404, the second end 1427 may be proximate the wearer’s pubic bone whereas the first end 1425 may be proximate the wearer’s perineum. In this configuration, the first end 1425 may be lower than the second end 1427, which may facilitate removal of fluid from the chamber 1404.

In some embodiments, the fluid collection device 1400 may further include a liner 1436 at a perimeter of the opening 1406. The liner 1436 may allow for air flow and/or prevent leaks. The liner 1436 may further provide a comfortable fit and/or feeling against the skin of the wearer. The liner 1436 may include an open cell foam in some embodiments. In some embodiments, the liner 1436 may include an adhesive on an edge 1438 adjacent to the wearer’s skin when in use to maintain the position of the fluid collection device 1400 on the wearer. In other embodiments, the fluid collection device 1400 may be held in place by mesh pants (not shown) worn by the wearer over the fluid collection device 1400.

Although not shown in FIG. 14, in some embodiments, the fluid collection device 1400 may further include a fluid permeable body positioned at least partially within the chamber 1404 and at least partially exposed to a wearer’s skin by the opening 1406. In some embodiments, the fluid permeable body may include a fluid permeable membrane and/or support. The fluid permeable membrane and/or support may be implemented in whole or in part by any or all of the embodiments of fluid permeable membranes and supports described herein, for example, those described with reference to FIGS. 1-13, except for suitable alterations in the shape and size to conform to the chamber 1404. In some embodiments, the fluid permeable body may only partially fill the chamber 1404, which may provide space within the chamber 1404 to accept the wearer’s anatomy. Similar to fluid permeable body 120, the fluid permeable body included in fluid collection device 1400 may wick fluids away from the wearer and facilitate fluid flow through the chamber 1404 to the conduit 1408 for removal from the fluid collection device 1400.

Although fluid collection device 1400 has been described in the example above with reference to a female wearer, in some applications, the fluid collection device 1400 may be suitable for a male wearer. For example, the chamber 1404 may be sized to accept male anatomy. In another example, the fluid impermeable barrier 1402 may be shaped to be placed proximate to buried and/or micro anatomy of male wearers.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

We claim:

1. A fluid collection device, comprising:
a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra; and
a fluid permeable body positioned at least partially within the chamber to extend across at least a portion of the opening and configured to wick fluid away from the opening, wherein the fluid permeable body includes a hydrophilic fluid permeable membrane and a support at least partially covered by the hydrophilic fluid permeable membrane.

2. The fluid collection device of claim 1, wherein the hydrophilic fluid permeable membrane includes nonwoven polypropylene.

3. The fluid collection device of claim 1, wherein the hydrophilic fluid permeable membrane includes nonwoven polyethelene.

4. The fluid collection device of any one of claims 1-3, wherein the hydrophilic fluid permeable membrane includes tapered pores, wherein a portion of the tapered pores proximate to the opening are wider than a portion of the tapered pores adjacent to the support.

5. A fluid collection device, comprising:
a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra; and
a fluid permeable body positioned at least partially within the chamber to extend across at least a portion of the opening and configured to wick fluid away from the opening, wherein the fluid permeable body includes a support at least partially covered by a fluid permeable membrane including a wicking fabric, wherein the wicking fabric is configured to wick the fluid from an outer surface of the fluid permeable membrane to an inner surface of the fluid permeable membrane.

6. The fluid collection device of claim 5, wherein the wicking fabric includes NANOtex Dry Inside.

7. The fluid collection device of claim 5 or 6, wherein the inner surface of the fluid permeable membrane is adjacent to the support and the outer surface of the fluid permeable membrane is opposite the inner surface of the fluid permeable membrane.

8. A fluid collection device, comprising:
a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra; and
a fluid permeable body positioned at least partially within the chamber to extend across at least a portion of the opening and configured to wick fluid away from the opening, wherein the fluid permeable body includes a support including a wicking portion proximate the opening and a packing portion proximate an interior surface of the chamber.

9. The fluid collection device of claim 8, wherein the packing portion is impermeable to at least one of the fluid or air.

10. The fluid collection device of claim 9, wherein the packing portion includes a closed cell foam.

11. The fluid collection device of any one of claim 8-10, wherein the wicking portion includes approximately a quarter of a circumference of the fluid permeable body.

12. The fluid collection device of any one of claims 8-10, wherein the wicking portion includes approximately one half of a circumference of the fluid permeable body.

13. The fluid collection device of any one of claims 8-10, wherein the wicking portion includes approximately one third of a circumference of the fluid permeable body.

14. The fluid collection device of any one of claims 8-13, wherein the wicking portion includes an open cell foam.

15. The fluid collection device of any one of claims 8-14, wherein the wicking portion includes a filter medium.

16. The fluid collection device of any one of claims 8-15, wherein the wicking portion includes a capillary foam.

17. A fluid collection device, comprising:
a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra; and
a fluid permeable body positioned at least partially within the chamber to extend across at least a portion of the opening and configured to wick fluid away from the opening, wherein the fluid permeable body includes a support including:
a wicking portion proximate to the opening;
a packing portion proximate an interior surface of the chamber; and
a sparger tube disposed in a center of the fluid permeable body, wherein the sparger tube extends at least a length of the fluid permeable body along a long axis of the fluid permeable body.

18. The fluid collection device of claim 17, wherein the wicking portion includes at least one opening for wicking the fluid and the sparger tube includes at least one second opening for wicking the fluid, wherein the at least one second opening is smaller than the at least one opening of the wicking portion.

19. The fluid collection device of claim 17 or 18, wherein the packing portion is impermeable to at least one of the fluid or air.

20. The fluid collection device of any one of claims 17-19, wherein the wicking portion includes an open cell foam.

21. The fluid collection device of any one of claims 17-20, wherein the sparger tube includes a porous polymer tube or a porous metal tube.

22. The fluid collection device of any one of claims 17-21, wherein the wicking portion includes approximately three quarters of a circumference of the fluid permeable body.

23. The fluid collection device of any one of claims 17-21, wherein the wicking portion includes approximately one half of a circumference of the fluid permeable body.

24. The fluid collection device of any one of claims 17-21, wherein the wicking portion includes approximately two thirds of a circumference of the fluid permeable body.

25. A fluid collection device, comprising:
a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra; and
a fluid permeable body positioned at least partially within the chamber to extend across at least a portion of the opening and configured to wick fluid away from the opening, wherein the fluid permeable body includes a support including a first layer and a second layer disposed concentrically about the first layer, wherein the first layer and the second layer have at least one of a different porosity or density.

26. The fluid collection device of claim 25, wherein a density of the first layer is greater than a density of the second layer.

27. The fluid collection device of claim 25, wherein a density of the first layer is less than a density of the second layer.

28. The fluid collection device of any one of claims 25-27, wherein a porosity of the first layer is greater than a porosity of the second layer.

29. The fluid collection device of any one of claims 25-27, wherein a porosity of the first layer is less than a porosity of the second layer.

30. The fluid collection device of any one of claims 25-29, wherein the fluid permeable body further comprises a fluid permeable membrane covering at least a portion of the second layer.

31. A fluid collection device, comprising:
a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra; and
a fluid permeable body positioned at least partially within the chamber to extend across at least a portion of the opening and configured to wick fluid away from the opening, wherein the fluid permeable body includes a support including a material having at least one of a density or a porosity gradient along a short axis of the fluid permeable body.

32. The fluid collection device of claim 31, wherein the material has a greater density at a location proximate to a center of the fluid permeable body and a lower density at a location proximate to an outer surface of the fluid permeable body.

33. The fluid collection device of claim 31, wherein the material has a lower density at a location proximate to a center of the fluid permeable body and a greater density at a location proximate to an outer surface of the fluid permeable body.

34. The fluid collection device of any one of claims 31-33, wherein the material has a greater porosity at a location proximate to a center of the fluid permeable body and a lower porosity at a location proximate to an outer surface of the fluid permeable body.

35. The fluid collection device of any one of claims 31-33, wherein the material has a lower porosity at a location proximate to a center of the fluid permeable body and a greater porosity at a location proximate to an outer surface of the fluid permeable body.

36. A fluid collection device, comprising:
a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra; and
a fluid permeable body positioned at least partially within the chamber to extend across at least a portion of the opening and configured to wick fluid away from the opening, wherein the fluid permeable body includes a support including a material having at least one of a density or a porosity gradient along a long axis of the fluid permeable body.

37. The fluid collection device of claim 36, wherein the support has a higher density at a location proximate the first end region of the fluid impermeable barrier and a lower density at a location proximate the second end region of the fluid impermeable barrier.

38. The fluid collection device of claim 36 or 37, wherein the support has a lower porosity at a location proximate the first end region of the fluid impermeable barrier and a higher porosity at a location proximate the second end region of the fluid impermeable barrier.

39. The fluid collection device of any one of claims 36-38, wherein the support comprises a plurality of annular-shaped portions, wherein each of the plurality of annular-shaped portions comprises a different density or porosity than others of the plurality of annular-shaped portions.

40. A fluid collection device, comprising:
a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra; and
a fluid permeable body positioned at least partially within the chamber to extend across at least a portion of the opening and configured to wick fluid away from the opening, wherein the fluid permeable body includes a fluid permeable membrane including a plurality of slot valves.

41. The fluid collection device of claim 40, wherein each of the plurality of slot valves comprises a pair of folds and a slit disposed between the folds.

42. The fluid collection device of claim 40 or 41, further comprising a conduit coupled to the aperture, wherein the conduit provides a suction to the chamber.

43. The fluid collection device of claim 42, wherein the slot valves are maintained in a closed position at least in part by the suction.

44. The fluid collection device of any one of claims 40-43, wherein the slot valves are configured to open in the presence of bolus flow of the fluid.

45. The fluid collection device of any one of claims 40-44, wherein the slot valve is configured to remain in a closed position absent flow of the fluid.

46. A fluid collection device, comprising:
a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a female urethra; and
a fluid permeable body positioned at least partially within the chamber to extend across at least a portion of the opening and configured to wick fluid away from the opening, wherein the fluid permeable body includes a support including a plurality of spokes extending from a central portion of the fluid permeable body to an outer perimeter of the fluid permeable body along a short axis of the fluid permeable body, wherein the plurality of spokes define a plurality of spaces there between.

47. The fluid collection device of claim 46, wherein the support comprises a material impermeable to at least one of air or the fluid.

48. The fluid collection device of claim 46 or 47, wherein each of the plurality of spokes extends along a long axis of the fluid permeable body.

49. The fluid collection device of any one of claims 46-48, wherein the fluid permeable body further comprises a fluid permeable membrane at least partially covering the support.

50. A fluid collection device, comprising:
a fluid impermeable barrier having a concave inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier and configured to be positioned adjacent to a urethra,
wherein a perimeter of the opening defines two opposing U-type shaped portions coupled at an angle.

51. The fluid collection device of claim 50, further comprising a liner disposed at the perimeter of the opening.

52. The fluid collection device of claim 51, wherein the liner comprises an open cell foam.

53. The fluid collection device of claim 51 or 52, wherein a surface of the liner includes an adhesive.

54. The fluid collection device of any one of claims 50-53, further comprising a fluid permeable body positioned at least partially within the chamber to extend across at least a portion of the opening and configured to wick fluid away from the opening.
